(19) United States
(12) Patent Application Publication
(10) Pub. No.: US 2009/0022819 A1
(43) Pub. Date: Jan. 22, 2009

(54) BACTERIAL EXTRACTS CULTURED IN THERMAL WATERS FOR THE TREATMENT OF DRY SKIN

(75) Inventors: AUDREY GUENICHE, RUEIL MALMAISON (FR); LIONEL BRETON, VERSAILLES (FR)

Correspondence Address: BUCHANAN, INGERSOLL & ROONEY PC, POST OFFICE BOX 1404, ALEXANDRIA, VA, 22313-1404, US

(73) Assignee: L'OREAL, PARIS (FR)

(21) Appl. No.: 12/175,072

(22) Filed: Jul. 17, 2008

Related U.S. Application Data
(60) Provisional application No. 60/929,966, filed on Jul. 19, 2007.

(30) Foreign Application Priority Data
Jul. 17, 2007 (FR) 0756531

(51) Int. Cl. A61K 33/00; A61K 35/74; A61P 17/00

(52) U.S. Cl. 424/717; 424/780; 424/715

(57) ABSTRACT

Dryness of keratin materials, in particular dryness of the skin, and especially disorders associated with dry and/or hyposeborrheic skin are treated by administering to individuals afflicted therewith, thus effective amounts of at least one extract of a non-photosynthetic and non-fruiting filamentous bacterium cultured in a medium which includes at least one non-sulfurous mineral and/or thermal water, e.g., an extract derived from the bacterium Vitreoscilla filiformis, in particular the strain ATCC 15551, cultured in a medium enriched with water from La Roche Posay.

### CROSS-REFERENCE TO COMPANION APPLICATIONS

Companion U.S. patent application Ser. No. ______ [Attorney Docket No.1016800-001050]; application Ser. No. ______ [Attorney Docket No.1016800-001051]; and application Ser. No. ______ [Attorney Docket No.1016800-001052], filed concurrently herewith, each hereby also expressly incorporated by reference and each also assigned to the assignee hereof.

### CROSS-REFERENCE TO PRIORITY/PROVISIONAL APPLICATIONS

This application claims priority under 35 U.S.C. § 119 of FR 0756531, filed Jul. 17, 2007, and of U.S. Provisional Application No. 60/929,966, Jul. 19, 2007, each hereby expressly incorporated by reference and each assigned to the assignee hereof.

### BACKGROUND OF THE INVENTION

1. Technical Field of the Invention

The present invention relates to the administration of at least one extract of a non-photosynthetic and non-fruiting filamentous bacterium cultured on a medium comprising at least one non-sulfurous mineral and/or thermal water for preventing and/or treating dryness of keratin materials, in particular dryness of the skin, and especially treating disorders associated with dry and/or hyposeborrheic skin.

2. Description of Background and/or Related and/or Prior Art

The skin is comprised of three superimposed layers, from the surface to deep in the body: the epidermis, the dermis and the hypodermis and also comprises ancillary structures such as, in particular, sebaceous glands.

The skin essentially performs a barrier function with respect to the outside environment, which results from a complex organization and numerous factors. This function is based in particular on the quality of the epidermis which depends especially on the degree of surface hydrophobicity of the stratum corneum and on the balance from proliferation and differentiation of the epidermal keratinocytes.

A breakdown in the balance of the skin can manifest itself in various ways. It may in particular result in the treatment of inflammatory processes, of a disturbance of sebaceous function or of hyperkeratinization, and also in an increase in insensible water loss and more generally in dryness of the skin. These events negatively affect the comfort and/or the aesthetics of the skin. They are also capable of affecting the state of health of the epidermis.

Thus, impairment of the barrier function will promote abnormal penetration of pathogenic agents into the horny layer and induce an increased release of pro-inflammatory substances responsible for inflammatory disorders of the skin which cause itching and tautness, two characteristic symptoms of dry skin.

A first alternative for treating dry skin characterized by a deficiency in lipids constituting the barrier and/or the aqueous-lipid film is aimed at the topical administration of products suitable to restore the skin barrier. These products are generally wetting agents, capable of fixing water, film-forming agents for retaining water, agents capable of reconstructing the skin barrier, such as exogenous lipids constituting the intercellular cement and the sebum, for instance squalene, ceramides, fatty acids or else active agents like vitamin C, capable of stimulating the endogenous synthesis of epidermal lipids.

As previously indicated, dry skin may also be the result of and/or be associated with an endogenous insufficiency of sebum production by the sebaceous glands.

Together with sweat, sebum constitutes a natural moisturizer for the epidermis and makes it possible to increase the suppleness and strength thereof. It is essentially composed of a more or less complex mixture of lipids. Conventionally, the sebaceous gland produces squalene, triglycerides, aliphatic waxes, cholesterol waxes and, possibly, free cholesterol. It is the action of bacterial lipases which converts a varying portion of the triglycerides formed to free fatty acids.

Conventionally, a sebum content of less than 100 μg/cm², measured at the T zone of the face, by the method described in FR 2 368 708, can be considered to be characteristic of hyposeborrheic dry skin.

An example of hyposeborrheic dry skin, or of skin becoming so, is observed during skin aging. Thus, the manifestation of xerosis related to a sebum deficiency is very commonly observed in elderly individuals, and in particular individuals over the age of 50. Moreover, sebum production insufficiency may be induced, by certain pharmaceutical treatments such as those involving corticosteroids.

### SUMMARY OF THE INVENTION

The present invention features the administration of novel compounds useful for the prevention and/or treatment, in general, of disorders associated with dryness of keratin materials.

It has now unexpectedly been demonstrated that the topical application of an extract of non-fruiting filamentous bacteria cultured on a medium enriched with water from La Roche Posay proves to be most particularly effective, in particular in adults, for the treatment of dry skin. The extract of non-fruiting filamentous bacteria cultured on a medium enriched with water preferably from La Roche Posay advantageously exhibits a beneficial activity on the skin barrier and limits dehydration of the skin.

Thus, the present invention features the cosmetic administration/topical application of at least one extract of a non-photosynthetic and non-fruiting filamentous bacterium cultured on a medium comprising at least one non-sulfurous thermal and/or mineral water, as an agent for treating the signs associated with dryness of keratin materials.

For the purpose of the present invention, the term “keratin material” is the skin, the scalp, the mucous membranes and semi-mucous membranes, the nails, and keratin fibers of human or animal origin.

### DETAILED DESCRIPTION OF BEST MODE AND SPECIFIC/PREFERRED EMBODIMENTS OF THE INVENTION

Bacterial extract:

The bacterial extracts according to the present invention are prepared according to a process comprising the culturing of at least one non-photosynthetic and non-fruiting filamentous bacterium in a medium comprising at least one non-sulfurous mineral and/or thermal water.

The bacteria are non-photosynthetic filamentous bacteria which comprise, in particular, the bacteria belonging to the order of the Beggiatoales and more particularly the bacteria belonging to the genera Beggiatoa, Vitreoscilla, Flexithrix or Leucothrix.

For implementing the invention, bacteria belonging to the genus Vitreoscilla are preferred, in particular bacteria of the species Vitreoscilla filiformis.

These bacteria, several of which have already been described, generally have an aquatic habitat and can be found in particular in sea waters or in thermal waters. Exemplary bacteria include:
Vitreoscilla filiformis (ATCC 15551)
Vitreoscilla beggiatoides (ATCC 43181)
Beggiatoa alba (ATCC 33555)
Flexithrix dorotheae (ATCC 23163)
Leucothrix mucor (ATCC 25107)
Sphaerotilus natans (ATCC 13338)

Preferably, the bacterium is that corresponding to the strain deposited at the ATCC under No.15551.

The term “thermal water” means a hot or cold water which is used for its therapeutic powers or for a bathing use. It is possible to use a thermal water or a mineral water. Generally, a mineral water is suitable for consumption, which is not always the case with a thermal water. Each of these waters comprises, inter alia, dissolved minerals and trace elements. These waters are known to be employed for specific treatment purposes depending on the particular trace elements and minerals present therein.

Preferably, a thermal and/or mineral water is employed which exhibits a total mineral content of greater than or equal to 400 mg/l.

According to this invention, the term “total mineral content” means the sum of the concentrations of anions and cations present in the thermal or mineral water. In the thermal or mineral waters according to the invention, the total mineral content generally ranges from 400 to 900 mg/l.

The thermal and/or mineral water according to the invention can have a total mineral content of at least 700 mg/l, in particular a total concentration of carbonates and of bicarbonates of at least 150 mg/l and more preferably of at least 360 mg/l and in particular of sodium carbonate and bicarbonate of greater than 2 mg/l. The concentration of silicon oxide in the water used in the composition according to the invention can preferably be at least 6 mg/l and more preferably at least 9 mg/l.

The thermal water or the mineral water according to the invention can be selected from water from Vittel, waters from the Vichy basin, water from Uriage, water from La Roche Posay, water from La Bourboule, water from Enghien-les-Bains, water from Saint-Gervais-les-Bains, water from Néris-les-Bains, water from Allevard-les-Bains, water from Digne, water from Maizières, water from Neyrac-les-Bains, water from Lons-Ie-Saunier, water from Eaux-Bonnes, water from Rochefort, water from Saint Christau, water from Les Fumades and water from Tercis-les-Bains.

Among these waters, those which exhibit a total concentration of carbonates or bicarbonates of greater than 360 mg/l are water from Vittel, water from La Bourboule, water from Les Fumades, water from Enghien-les-Bains, water from La Roche Posay, water from the Vichy basin and water from Uriage.

Among these waters, those which exhibit a concentration of carbonates or bicarbonates of from 150 mg/l and 360 mg/l are water from Digne, water from Maizières, water from Rochefort or water from Saint Gervais-les-Bains.

Among these waters, those which comprise at least 2 mg/l of sodium carbonate or bicarbonate are water from La Roche Posay, water from Vittel, waters from the Vichy basin or water from Uriage.

The waters comprising at least 9 mg/l of silicon oxide are water from La Roche Posay, water from Vittel, waters from the Vichy basin or water from Uriage.

The thermal or mineral waters which are particularly suitable for the implementation of the invention have a concentration of calcium ions of greater than or equal to 100 mg/l, indeed even 140 mg/l.

According to one advantageous embodiment, the thermal or mineral water has a concentration of hydrogencarbonate ions of greater than or equal to 300 mg/l. The hydrogencarbonates, also known as bicarbonates, are present in particular at a concentration of greater than or equal to 350 mg/l.

According to another advantageous embodiment, the bacteria are cultured in a medium comprising at least one thermal water. The latter can in particular be selected from water from Vittel, waters from the Vichy basin, water from Uriage, water from La Roche Posay, water from La Bourboule, water from Les Fumades, water from Enghien-les-Bains or water from Eaux-Bonnes.

The waters which make it possible to obtain a particularly advantageous result according to the invention are selected in particular from water from La Roche Posay and water from Vittel, or a water with a similar composition.

Water from La Roche Posay is extracted from the spring of the same name; it is a water comprising bicarbonate, calcium, silicate and selenium. It generally comprises approximately 387 mg/l of bicarbonate ions, approximately 140 mg/l of calcium ions and at least 4 mg/l of sulfates.

Water from Vittel is rich in calcium and in mineral salts (841 mg/l) and comprises in particular 202 mg/l of calcium, 402 mg/l of bicarbonates and 336 mg/l of sulfates.

Culturing can in particular be carried out in the following medium:

| Composition: | Concentration: | |
|---|---|---|
| Autolyzed yeast extract | 0.5 to 5 | g/l |
| Plant peptone | 0.5 to 5 | g/l |
| Anhydrous glucose | 0.5 to 7 | g/l |
| Heller microelements | 0.5 to 5 | ml/l |
| CaCl₂•10H₂O | 0.010 to 0.200 | g/l |

The composition is made up to 1,000 ml with mineral and/or thermal water optionally topped with distilled or osmosed water.

Exemplary peptones include soybean papain peptone.

This medium is distinguished from the media generally used by the absence of catalase and sulfide.

The Heller microelements have been described by Heller, Ann. Sci. Nat. Biol. Veg., 14, 1-223 (1953). They are mixtures of various mineral elements which are recommended by Heller not for the culturing of bacteria but for the nutrition of plant tissues cultured in vitro.

Culturing can be carried out at the appropriate temperature suitable for the bacterial species cultured. Generally, this temperature ranges from 18 and 40° C., depending on the strains. The pH of the culture medium preferably ranges from 5.5 to 8.

The composition of the Heller microelements, per 1 l of water, is as follows:

| | | |
|---|---|---|
| ZnSO₄•7H₂O | 1 | g |
| MnSO₄•H₂O | 0.076 | g |
| CuSO₄•5H₂O | 0.003 | g |
| Kl | 0.010 | g |
| H₃BO₃ | 1 | g |
| AlCl₃•6H₂O | 0.050 | g |
| NiCl₂•6H₂O | 0.030 | g |

Said thermal or mineral waters can replace all or part of the aqueous phase of the culture medium. They can thus be a mixture in any proportion with the water, in particular distilled or osmosed water, present in the culture medium. The mixture (i) of thermal water and (ii) of osmosed or distilled water could be in a ratio from 0.1% to 100%, especially from 0.1 to 50, in particular from 0.1 to 25.

After mixing all the elements of the medium, the culture medium comprising the thermal and/or mineral water is advantageously sterilized; this stage is carried out by methods known to one skilled in the art, such as sterilization by filtration or by heat.

The culture medium is subsequently inoculated with the bacteria.

The media most suitable for culturing bacteria are such that the thermal or mineral water preferably is at least 0.1% of the amount of water introduced for the preparation of the medium, in particular from 0.1 to 99.9%. Good results are obtained with concentrations of thermal water of approximately 1.33%, with respect to the osmosed and/or distilled water, for example from 0.5 to 20%, indeed even from 0.5 to 50%, but these concentrations can be increased without disadvantage.

In known fashion, the process for preparing the bacterial extract comprises at least one stage in which the bacteria are recovered at the end of culturing, in particular by separating them from the culture medium.

After culturing the bacteria, the biomass can be isolated by various known methods, for example by filtration, by coagulation with an alcohol (ethanol, isopropanol, isobutanol), by drying on a cylinder with a scraped precoat (starch, diatoms, and the like) or by freeze-drying. A preliminary concentration, for example at 80° C. under reduced pressure, improves this separation.

The biomass may be used alive or else be treated by various processes. An operation of rupturing the envelopes can be carried out, for example by the action of ultrasound. In addition, extracts can be prepared using an alcohol, such as ethanol or propanol.

Lipopolysaccharide extracts can also be prepared according to known methods; for example, see Noris and Ribbons, Methods in Microbiology, Vol. 5B, Academic Press (1971). The method generally used is the well-known “Westphal” method (or a related method), which consists in carrying out the extraction with phenol/water mixtures at 65° C. The extract is subsequently subjected to dialysis in order to remove the phenol.

The bacterial extract employed according to the invention may also result from the implementation of the following process: (i) at least one bacterium belonging to the order of the Beggiatoales is cultured in a medium comprising a monosaccharide as main carbon source and at least one mineral or thermal water and then (ii), after fermentation, the bacteria are separated from the culture medium in order to recover said mass of bacteria.

The bacteria recovered on conclusion of the fermentation stage can in particular be subjected to a stabilization and/or extraction treatment. It is the extract of filamentous bacteria which is thus obtained which will generally be used in or for the preparation of cosmetic or dermatological compositions. In a way known per se, the extract can thus be sterilized, in particular by filtration or by autoclaving.

The term “extract of non-photosynthetic filamentous bacteria” means equally well the supernatant from the culturing of said bacteria, the biomass obtained after culturing said bacteria or the extracts of the biomass which are obtained by treatment of this biomass.

In order to prepare the extracts according to the invention, said bacteria can be cultured according to the above process and can then be separated from the biomass obtained, for example by filtration, centrifuging, coagulation and/or freeze-drying.

Thus, after culturing, the bacteria are concentrated by centrifuging. The biomass obtained is autoclaved. This biomass can be freeze-dried in order to constitute what is referred to as the freeze-dried extract. Any freeze-drying method known to one skilled in the art can be used to prepare this extract.

The supernatant fraction from this biomass can also be filtered into a sterile container in order to remove the suspended particles. This supernatant fraction can also be decanted under sterile conditions into a sterile container. According to a specific embodiment of the invention, the supernatant fraction thus obtained is used as cosmetic or dermatological active principle.

The bacterial extracts according to the invention may be formulated in a suitable carrier in an amount of at least 20% by weight relative to the total weight of the composition, in particular in an amount of 0.001 to 20% by weight relative to the total weight of the composition and more particularly in an amount of 0.01 to 10% by weight relative to the total weight of the composition.

For certain applications or specific formulations, it may be advantageous to use high weight concentrations of bacterial extract, for example from 15 and 20%.

The bacterial extract cultured in a medium enriched with thermal water may also be used in the form of fractions of cellular components or in the form of metabolites. The microorganism(s), metabolite(s) or fraction(s) may also be introduced in the form of a freeze-dried powder, a culture supernatant and/or, where appropriate, in a concentrated form.

For certain applications, the living biomass may be used as is, for example in the form of masks or a poultice for producing an immediate effect.

According to the invention, the term “metabolite” is any substance derived from the metabolism of the microorganisms considered according to the invention and endowed with an efficacy for treating dark circles.

Unexpectedly, it has now been observed that the bacterial extracts cultured in thermal water were able to prove effective for regulating vascularization defects of the contour of the eyes and thus to prevent and/or reduce bags and/or dark circles around the eyes.

Specifically, it has now been demonstrated that the extract of the bacterium Vitreoscilla filiformis cultured in thermal water from La Roche Posay has an increased effectiveness in treating vascular disorders compared to the extract of the same bacterium cultured in a conventional medium, that is to say, without mineral or thermal water.

The main difference from these two extracts is in the procedures for preparing the culture medium where there is substitution of osmosed water by water from La Roche Posay. This leads in particular to a modification of the metabolism of the bacteria caused by an enrichment of the culture medium in mineral elements, particularly in selenium, strontium and zinc.

It is also interesting to note that the introduction of this biomass into a formulatory carrier does not present a risk of overexposure to these elements, since Se and Zn are elements that are essential to the body and Sr is widespread in food.

The table below provides the concentrations of these chemical elements in the bacterial extract according to the invention prepared according to the procedure of Example 1 (freeze-dried extract).

| | |
|---|---|
| Se (mg/kg) | 6 |
| Sr (mg/kg) | 10 |
| Zn (mg/kg) | 216 |

Thus, the application of this enriched extract leads to topical exposures of mineral salts per day of around:

| | |
|---|---|
| Se (μg/day) | 0.008 |
| Sr (μg/day) | 0.0032 |
| Zn (μg/day) | 0.094 |

It is noted here that the use of ions for improving skin condition is very old. Thus, dermatologically-targeted thermal cures on the banks of the Dead Sea—the saltiest expanse of water in the world—go back to ancient times (Abels D J et col., Clinics in Dermatol., 14: 653-658,1996). These baths exert an anti-pruriginous activity and it is not uncommon that people treated experience the feeling of having smoother and more supple skin (Even-Pazz Z, Isr J. Med. Sci., 32: 11-15,1996). To date, the advantage of the topical application of cations has been studied as much in the field of sensitivity as in that of skin dryness. Among the divalent cations, it is the calming effect of strontium which has been most documented (Hahn G S, In biochemical modulation of skin reactions. Kydonieus A F, Will J J (eds.), CRE, Boca Raton, Fla., US, 261-272, 2000).

Thus, the present invention features the cosmetic administration of at least one extract of a non-photosynthetic and non-fruiting filamentous bacterium cultured in a medium comprising at least one non-sulfurous thermal and/or mineral water, as an agent useful for treating the signs associated with dryness of keratin materials.

Unless otherwise indicated, the term “treating” means any action useful for improving the comfort or the well-being of an individual, this term therefore equally covers preventing, attenuating, reducing, relieving and curing.

In particular, said bacterial extracts can be administered as an agent:
for treating states of dryness of the skin, skin which has a rough appearance and/or which is rough to the touch; squamous states; in particular dandruff conditions;
for treating dry skin;
for treating itching and/or tautness associated with dry skin;
for treating skin disorders related to a deficiency in excretion and/or secretion of sebum;
for physiologically restoring a suitable state of hydration of the stratum corneum;
for treating hyposeborrheic dry skin;
for stimulating sebum production;
for treating dry keratin fibers;

In the case of human or animal keratin fibers, like the hair, body hairs and/or eyelashes, the bacterial extracts according to the invention are particularly advantageous for preventing and/or treating the manifestation of signs of fragility, for instance dryness which is generally reflected by the fiber having a brittle aspect. The extract thus makes it possible to confer a shiny appearance on keratin fibers, in particular the human head of hair and the coat of animals:
for treating functional disorders of the pilosebaceous unit;
for preventing and/or reducing wrinkles related to dryness of the skin;
for improving the comfort of dry skin and a dry scalp;
for combating the dull and/or lifeless appearance of the skin and/or of the hair resulting from them drying out.

Dryness of the skin is often associated with a decrease in the degree of hydration of the skin, evaluated by corneometry, and with an impairment of the barrier function, measured by insensible water loss.

Dry skin essentially manifests itself through a feeling of tautness and/or of tension. Said dry skin is also rough to the touch and appears to be covered with squames. When the skin is slightly dry, the squames are abundant but barely visible to the naked eye. When this disorder worsens, they become fewer and fewer in number but increasingly visible to the naked eye.

The cause of dryness of the skin may be of constitutional or acquired type.

In the case of acquired dry skin, the involvement of outside parameters such as exposure to chemical agents, to difficult climatic conditions or to sunlight, alternatively certain therapeutic treatments (retinoids, for example) is determinant. Under these outside influences, the skin may then become momentarily and locally dry. This can involve any type of normal and even oily skin.

In the case of constitutional dry skin, two categories can be distinguished: pathological skin and non-pathological skin.

Pathological constitutional dry skin is essentially represented by atopic dermatitis and ichthyoses. It is virtually independent of the outside conditions.

Atopic dermatitis is described as being associated with a deficiency in metabolism of the lipids of the stratum corneum, and in particular of the ceramides. This pathology presents itself in the form of more or less chronic xerosis involving a large extent of the body, associated with inflammatory and pruriginous exacerbations in plaques.

Icthyoses are pathologies characterized by a genetic deficiency that affects the keratinization process at various stages. It manifests itself through considerable desquamation in plaques.

Non-pathological constitutional dry skin is dry skin for which the severity can depend on the outside factors already indicated. Senile skin (characterized by a general decrease in metabolism in the skin with age), fragile skin (very sensitive to outside factors and often accompanied by erythema and rosacea) and common xerosis (of probable genetic origin and manifesting itself predominantly on the face, the limbs and the back of the hands) enter into this skin category.

The administration according to the invention is thus found to be particularly effective for preventing and/or treating dry skin, and more particularly acquired dry skin and/or constitutional dry skin.

The bacterial extracts according to the invention are therefore useful for the preparation of compositions for use in the prophylactic or therapeutic treatment of atopic dermatitis (in the remission phases as a maintenance treatment), atopic xerosis, seborrheic dermatitis and hydrokeratosis.

The present invention also features extracts of non-photosynthetic and non-fruiting filamentous bacteria cultured on a medium comprising at least one non-sulfurous thermal and/or mineral water, for administration in the prophylactic or therapeutic treatment of atopic dermatitis (in the remission phases as a maintenance treatment), atopic xerosis, seborrheic dermatitis and hyperkeratosis.

The bacterial extracts according to the invention may be advantageously combined with other active agents.

Exemplary such active agents include vitamins B3, B5, B6, B8, C, E or PP, carotenoids, curcuminoids and niacin.

Administration according to the invention is carried out by any route suitable for the desired effect, in particular orally or topically, advantageously topically onto the skin.

The term “topical administration” means administration of the extracts according to the invention or of the compositions containing it by application to the skin as defined above.

Unless otherwise indicated, the term “skin” means any cutaneous surface of the body, including the skin and extended to the scalp and the mucous membranes and semi-mucous membranes, and the term “appendages” means the eyelashes, body hair, head hair and nails.

The topical compositions according to the invention which are useful for treating the skin, the mucous membranes and semi-mucous membranes and the scalp may be in the form of salves, creams, milks, ointments, powders, impregnated pads, solutions, gels, sprays, lotions or suspensions. They may also be in the form of lipid or polymeric vesicules or nanospheres or microspheres or polymeric patches and hydrogels for controlled release. These topical compositions may be either in an anhydrous form or in an aqueous form according to the dermocosmetic indication.

The compositions are in particular for use in hair hygiene. They may be in particular in the form of a cream, a milk, a lotion, a gel, lipid or polymeric vesicules or nanospheres or microspheres, a soap or a shampoo.

The present invention also features compositions combining at least one bacterial extract in combination with other active agents.

It is advantageous to introduce into the composition according to the invention at least one compound selected from: desquamating agents; moisturizing agents; anti-inflammatories or calmatives; agents for stimulating keratinocyte proliferation and/or differentiation; anti-dandruff agents; and anti-microbial agents.

Indeed, the stimulation of seborrhea with the bacterial extracts according to the invention may, in certain individuals, provide a proliferation terrain for the resident microflora of the follicular ostium (in particular Propionibacterium acnes), thus resulting in considerable hydrolysis of the sebum triglycerides to free fatty acids and the reduction of the unsaturations of polyunsaturated fatty acids (linoleic acid in particular). These two phenomena may contribute towards keratinization of the infundibulum and the formation of a microcomedone. This may degenerate into a comedone, plugging and dilating the pore in an unattractive manner. At a more advanced stage, this plug may diverge towards an inflammatory acneic lesion.

The addition of desquamating agents or agents for stimulating keratinocyte proliferation or differentiation to the compositions according to the invention makes it possible to avoid the formation of these comedones. Similarly, anti-microbial, anti-bacterial or bacteriostatic agents make it possible, by modifying the proliferation of the resident microflora, to obtain the same effect.

In addition, moisturizing agents may supplement the effect obtained using the bacterial extracts according to the invention, and calmatives can be included to improve the comfort of oligoseborrheic dry skin.

Finally, the use of anti-dandruff agents is advantageous when the composition according to the invention is for the treatment of dry scalps.

Desquamating agent:

The term “desquamating agent” means any compound capable of acting:
either directly on desquamation by promoting exfoliation, such as β-hydroxy acids, in particular salicylic acid and its derivatives (including 5-n-octanoyl salicylic acid); α-hydroxy acids, such as glycolic acid, citric acid, lactic acid, tartaric acid, malic acid or mandelic acid; urea; gentisic acid; oligofucoses; cinnamic acid; extract of Saphora japonica; resveratrol and certain jasmonic acid derivatives;
or on the enzymes involved in desquamation or degradation of corneodesmosomes, glycosidases, stratum corneum chymotryptic enzyme (SCCE), or even other proteases (trypsin, chymotrypsin-like). Mention may be made of agents for chelating mineral salts: EDTA; N-acyl-N,N′,N′-ethylenediaminetriacetic acid; aminosulfonic compounds and in particular (N-2-hydroxyethylpiperazine-N-2-ethane)sulfonic acid (HEPES); derivatives of 2-oxothiazolidine-4-carboxylic acid (procysteine); derivatives of alpha-amino acids of glycine type (as described in EP 0 852 949, and also the sodium methyl glycene diacetate marketed by BASF under the trademark TRILON M); honey; sugar derivatives such as O-octanoyl-6-D-maltose and N-acetylglucosamine.

Moisturizing agent:

The term “moisturizing agent” means:
either a compound that acts on the barrier function, with a view to maintaining the moisturization of the stratum corneum, or an occlusive compound. Mention may be made of ceramides, sphingoid-based compounds, lecithins, glycosphingolipids, phospholipids, cholesterol and its derivatives, phytosterols (stigmasterol, β-sitosterol, campesterol), essential fatty acids, 1,2-diacylglycerol, 4-chromanone, pentacyclic triterpenes such as ursolic acid, petroleum jelly and lanolin;
or a compound that directly increases the water content of the stratum corneum, such as threalose and its derivatives, hyaluronic acid and its derivatives; glycerol, pentanediol, sodium pidolate, serine, xylitol, sodium lactate, polyglycerol acrylate, ectoin and its derivatives, chitosan, oligosaccharides and polysaccharides, such as the product marketed under the reference Pentavitin, honey, alginates (in particular the product Sobalg PH 154 marketed by Grindsted), cyclic carbonates, N-lauroylpyrrolidonecarboxylic acid or its salts, in particular the sodium salt marketed under the reference Nalidone, and N-α-benzoyl-L-arginine;
or a compound that activates the sebaceous glands, such as steroid derivatives (including DHEA, its 7-oxide and/or 17-alkyl derivatives and sapogenins), methyl dihydrojasmonate, and vitamin D and its derivatives.

These compounds may represent from 0.001% to 30%, and preferably from 0.01 to 20%, of the total weight of the composition according to the invention.

Anti-glycation agent:

The term “anti-glycation agent” means a compound that prevents or decreases the glycation of the proteins of the skin, in particular the proteins of the dermis, such as collagen.

Examples of anti-glycation agents are extracts of plants of the family Ericaceae, such as an extract of blueberry (Vaccinium angustifolium); ergothioneine and its derivatives; and hydroxystilbenes and their derivatives, such as resveratrol and 3,3′,5,5′-tetrahydroxystilbene. These anti-glycation agents are described in FR 99/16166, FR 00/08158, FR 99/09267 and FR 99/16168, respectively. Resveratrol is particularly preferred for use in this invention.

The compositions according to the invention comprising an anti-glycation agent as defined above can advantageously be used for preventing or treating the signs of skin aging, in particular for preventing or treating the loss of tonicity and/or elasticity of the skin.

Anti-inflammatories and/or culmatives:
an antagonist of inflammatory cytokines;
a steroidal anti-inflammatory (hydrocortisone, betamethasone, dexamethasone, etc.);
a non-steroidal anti-inflammatory such as aspirin or paracetamol;
beta-glycyrrhetinic acid, extracts containing it, for instance the extract of Glycyrrhiza glabra (licorice) and complexes containing it, such as the allantoin/glycyrrhetinic acid complex;
planktons, which may or may not be lyophilized, extracts thereof and complexes thereof;
escin and plant extracts containing it, such as extract of horse chestnut;
xanthin derivatives, such as diethylaminoethyltheophylline hydrochloride and caffeine;
waters and extracts (for example, aqueous, aqueous-alcoholic or water-glycol extracts) of flowers and of plants, such as cornflower water, camomile water, mint water, lime blossom water or rose water, extracts of Rosaceae (for example: Rosa gallica), extracts of peony, extracts of hawthorn, extracts of yarrow, extracts of mallow, extracts of marigold, extracts of melilot, extracts of sage, extracts of elder, extracts of ginkgo biloba, extracts of arnica, extracts of oregano, extracts of green tea, extracts of waterlily blossom, extracts of birch bark, extracts of aloe vera;
asiatic acid and plant extracts containing it, such as Centella Asiatica;
fruit extracts, such as extract of pineapple, extract of papaya; extract of guava;
algae, in particular of the Laminaria type (for example, red or brown algae);
pyrrolidonecarboxylates, and in particular of zinc (Zn-PCA) or of copper (Cu-PCA);
oils of plant origin, such as canola seed oil and shea butter oil;
essential oils, for example of coriander, of balm, of lavender, of mint or of camomile, and mixtures thereof;
acexamic acid and transexamic acid (trans-4-aminomethylcyclohexanecarboxylic acid);
ursolic acid and extracts containing it, such as extract of rosemary leaf;
polysaccharides containing fucose, such as FUCOGEL 1000, marketed by Solabia (aqueous solution containing 1% of polysaccharide solids comprising fucose, galactose and galacturonic acid);
electrolytes, and in particular an aqueous mixture comprising from 30% to 35% of magnesium chloride, from 20% to 28% of potassium chloride, from 3% to 10% of sodium chloride, from 0.2% to 1% of calcium chloride, from 0.1% to 0.6% of magnesium bromide and from 0.1% to 0.5% of insoluble matter, said mixture being referred to herein as “Dead Sea Bath Salts” since it corresponds to the main salts contained in the Dead Sea;
galactolipids, for example from oat, for instance digalactosyl diglyceride or monogalactosyl diglyceride;
amino acids, derivatives thereof and salts thereof, such as the sodium salt of amino acids grafted onto cocoyl chains, marketed in the form of a mixture under the trademark SEPICALM S by SEPPIC, capryloylglycine marketed under the trademark LIPACIDE C8G by SEPPIC, and the mixture of capryloylglycine, cinnamon and sarcosine marketed under the trademark SEPICONTROL A5 by SEPPIC;
TNF-alpha antagonists, such as lisophylline, A802715, sulfasalazine, CDP-571 (anti-TNF-alpha antibody) or MDL-201112;
substance P antagonists, such as sendide, spantide II, and the peptides described in EP-A-680749, and the extracts of filamentous bacteria described in application EP-A-761204;
CGRP antagonists, such as CGRP 8-37, anti-CGRP antibodies or plant extracts with CGRP antagonist activity (for example: Iris pallida); divalent strontium, zinc, manganese, magnesium and calcium salts, such as those described in WO-A-96/19184, WO-A-96/19182 and WO-A-96/19228;
   and mixtures thereof.

The term “antagonists of inflammatory cytokines” according to the invention means a compound capable of inhibiting the synthesis and/or the release of one or more inflammatory cytokines. Compounds which inhibit or block the binding of the cytokines to their receptor(s) also come under the definition of an antagonist of inflammatory cytokines.

Exemplary are compounds that are antagonists of IL-1, of IL-8, of TNFα and of TNFβ, the tripeptide Lys-Pro-Val (KPV), and all the derivatives of αMSH and related peptidometics characterized by an activity of αMSH type through binding to the receptor or through control of the relase of IL1, of IL8 or of TNFα, all inhibitors of cytokine release (therapeutic class of the CSAIDs, for: cytokine suppressive anti-inflammatory drugs), the family of substituted pyrimidine N-oxides (EP99401719.2 (priority FR9809509) and EP 99402771.2 (priority FR9814211)) that are inducers of lipoxine A4, extracts of algae capable of modulating the production of cytokines by keratinocytes, such as Phycosaccharide® marketed by CODIF, Phlorogine® marketed by SECMA, extracts of Aloe vera or of Gingko biloba; the natural IL-1 antagonist (IL-1 RA).

Also exemplary are immunomodulator peptides, such as the Gly-Gln-Pro-Arg polypeptide and derivatives described in PCT/FR00/00031.

The anti-inflammatories are preferably present in the compositions in accordance with the invention at a concentration that may range from 0.00001% and 10% by weight approximately, relative to the total weight of the composition. Even more preferably, the concentration of anti-inflammatory compound may range from 0.0005% to 2% by weight, relative to the total weight of the composition.

Agent for stimulating fibroblast or keratinocyte proliferation and/or keratinocyte differentiation:

The agents for stimulating fibroblast proliferation that can be incorporated into the compositions according to the invention may, for example, be selected from among plant proteins or polypeptides, extracted in particular from soybean (for example, an extract of soybean marketed by LSN under the trademark Eleseryl SH-VEG 8® or marketed by SILAB under the trademark Raffermine®); and plant hormones such as giberrellins and cytokines.

The agents for stimulating keratinocyte proliferation which can be incorporated into the compositions according to the invention comprise in particular retinoids such as retinol and its esters, including retinyl palmitate; adenosine; phloroglucinol; extracts of walnut cakes marketed by GATTEFOSSE; and the extracts of Solanum tuberosum marketed by SEDERMA.

The agents for stimulating keratinocyte differentiation comprise, for example, minerals such as calcium; a peptide extract of lupin such as that marketed by SILAB under the trademark Structurine®; sodium beta-sitosteryl sulfate such as that marketed by SEPORGA under the trademark Phytocohesine®; and a water-soluble extract of maize such as that marketed by SOLABIA under the trademark Phytovityl®; a peptide extract of Voandzeia subterranea such as that marketed by Laboratoires Sérobiologiques under the trademark Filladyn LS 93970; and lignans such as secoisolariciresinol.

Anti-dandruff agents:

The anti-dandruff agents may be any active agent that can be used to prevent the appearance of dandruff, to reduce the number of times it occurs and/or to make it completely disappear. Thus, the anti-dandruff agent may be selected from among the anti-fungal and/or anti-bacterial agents indicated hereinafter.

More particularly, these agents may be selected from among:
pyridinethione salts in particular the calcium, magnesium, barium, strontium, zinc, cadmium, tin and zirconium salts. The zinc salt of pyridinethione is particularly preferred.

The zinc salt of pyridinethione, in particular marketed under the trademark zinc omadine by OLIN;
azol compounds, such as climbazole, ketoconazole, clotrinazole, econazole, isoconazole and miconazole;
anti-fungal polymers such as amphotericin B or nystatin;
selenium sulfides, in particular those of formula SₓSe₈₋ₓ, x ranging from 1 to 7;
other anti-dandruff agents, such are sulfur in its various forms, cadmium sulfide, allantoin, coal tar or wood tar and derivatives thereof, in particular oil of cade, salicylic acid, undecylenic acid, fumaric acid, allylamines such as terbinafine.

These are also agents for combating desquamative states of the scalp, which are preferably selected from among pyridinethione salts such as zinc pyrithione, 1-hydroxy-2-pyrrolidone derivatives such as piroctone and piroctone olamine; selenium sulfides such as selenium disulfide; climbazole, undecylenic acid; ketoconazole; cyclopirox, or mixtures thereof. In practice, these additional active agents or the mixture of additional active agents may represent from 0.001% to 10% by weight, relative to the total weight of the composition, and preferably from 0.1% to 5% by weight.

Antimicrobial agents:

The anti-microbial agents that can be incorporated into the compositions according to the invention may in particular be selected from among 2,4,4′-trichloro-2′-hydroxydiphenyl ether (or triclosan), 3,4,4′-trichlorobanilide, phenoxyethanol, phenoxypropanol, phenoxyisopropanol, hexamidine isethionate, metronidazole and its salts, miconazole and its salts, itraconazole, terconazole, econazole, ketoconazole, saperconazole, fluconazole, clotrimazole, butoconazole, oxiconazole, sulfaconazole, sulconazole, terbinafine, ciclopirox, ciclopiroxolamine, undecylenic acid and its salts, benzoyl peroxide, 3-hydroxybenzoic acid, 4-hydroxybenzoic acid, phytic acid, N-acetyl-L-cysteine acid, lipoic acid, azelaic acid and its salts, arachidonic acid, resorcinol, 2,4,4′-trichloro-2′-hydroxydiphenyl ether, 3,4,4′-trichlorocarbanalide, octopirox, octoxyglycerine, octanoylglycine, caprylyl glycol, 10-hydroxy-2-decanoic acid, dichlorophenylimidazole dioxolane and its derivatives described in WO 93/18743, farnesol and phytosphingosines, and mixtures thereof.

The preferred anti-microbial agents are triclosan, phenoxyethanol, octoxyglycerine, octanoylglycine, 10-hydroxy-2-decanoic acid, caprylyl glycol, farnesol and azelaic acid.

By way of example, the anti-microbial agent may be incorporated into the composition according to the invention in an amount representing from 0.1% to 20%, and preferably from 0.1% to 10%, of the total weight of the composition.

According to another of its embodiments, the present invention features a cosmetic regime or regimen for preventing and/or treating dry skin, comprising the administration, in particular topical administration, of an effective amount of at least one extract of non-photosynthetic and non-fruiting filamentous bacteria cultured on a medium enriched with water from La Roche Posay, one of their fractions or one of their metabolites.

The cosmetic treatment of the invention may be carried out in particular by applying the compositions as defined above, according to the customary technique for using these compositions. For example: applications of creams, gels, sera, lotions, milks for removing makeup or aftersun compositions to the skin or to dry hair, application of a hair lotion to wet hair, or of shampoo, or else application of toothpaste to the gums.

The cosmetic regime or regimen according to the invention can be carried out by topical administration. It may comprise a single application. According to another embodiment, the application is repeated, for example, two to three times daily over one day or more, and generally over a sustained period of at least four weeks, or even four to fifteen weeks, with, where appropriate, one or more periods of interruption.

In order to further illustrate the present invention and the advantages thereof, the following specific examples are given, it being understood that same are intended only as illustrative and in nowise limitative. In said examples to follow, all parts and percentages are given by weight, unless otherwise indicated.

### EXAMPLE 1

### Preparation of a Bacterial Extract According to the Invention: Biomass of Vitreoscilla filiformis Cultured on a Medium Enriched with Thermal Water from La Roche Posay

Preparation of the culture medium:

Composition:

| Yeast extract | 2 to 3 | g |
|---|---|---|
| Soybean papain peptone | 2 to 3 | g |
| Glucose | 2 to 3 | g |
| Heller microelements | 2 | ml |
| CaCl₂•2H2O | 66.21 | mg |
| Thermal water from La Roche Posay | 13-14 | ml |

This stock solution will be diluted with osmosed water in a ratio of 1/75 before sterilization.

The pH of the medium is adjusted to 5.00 by adding a molar solution of H₃PO₄. The medium is sterilized by autoclaving at 121° C. for 30 minutes. After cooling to ambient temperature, the pH is readjusted to 7.20 by adding a molar solution of KOH.

Culturing:

After the medium has been inoculated at 1% with the Vitreoscilla filiformis strain, the culture is shaken on an orbital shaker at 100 rpm and at 26° C. After growth for 48 hours, the culture is centrifuged at 8,000 g for 15 minutes. The pellets are recovered and then autoclaved at 121° C. for 30 minutes. This biomass can be used for evaluation tests.

### EXAMPLE 2

### Clinical Trials on Individuals Suffering from Atopic Dermatitis

Clinical data:

A first clinical study (study 1), double blind, evaluated intraindividually the comparative effect of a cream containing 5% of extract of Vitreoscilla filiformis (V. f.) cultured conventionally (hereinafter referred to as “conventional extract”) on the red blotches occurring in individuals suffering from slight to moderate atopic dermatitis (symmetrical lesions versus placebo).

The “conventional” extract of Vitreoscilla filiformis is prepared according to the following modes:

Preparation of the culture medium:

Composition:

| | | |
|---|---|---|
| Yeast extract | 2 | g |
| Soybean papain peptone | 2 | g |
| Glucose | 2 | g |
| Heller microelements | 2 | ml |
| CaCl₂•2H2O | 66.21 | mg |
| Water | qs 1 | l |

The pH of the medium is then adjusted to 5.00 by adding a molar solution of H₃PO₄. The medium is sterilized by autoclaving at 121° C. for 30 minutes. After cooling to ambient temperature, the pH is readjusted to 7.20 by adding a molar solution of KOH.

Culturing:

In the laboratory: after the medium has been inoculated at 1% with the Vitreoscilla filiformis strain, the culture is shaken on an orbital shaker at 100 rpm and at 26° C. After growth for 48 hours, the culture is centrifuged at 8,000 g for 15 minutes. The pellets are recovered and then autoclaved at 121° C. for 30 minutes. This biomass can be used for evaluation tests.

In fermenter:

In a fermenter preferably equipped with a draft tube in order to limit the shear force, the V. f. strain is inoculated at a minimum of 1% volume. The pH is kept stable at 7 UpH throughout the culturing, the T° is adjusted from 26 to 28° C. and the oxygenation is maintained at 10% pO2 throughout the culturing, by the action either of the shaking speed or by adjustment of the air flow rate. This type of culturing can be carried out in batch, fed-batch or in continuous mode. The latter technique, which guarantees a reproducible biomass through controlling the growth rate (μ), will be preferred. The biomass harvested continuously by centrifugation at 10,000 g is frozen at −20° C. When the freezing tank is full, it is thawed at 4° C. and then packaged in packages that can be handled by an operator. These packages containing the biomass are then sterilized in order to be stabilized. This sterilization operation then is a production batch.

In this study, the products containing the extract applied twice a day were very well tolerated.

The extract is formulated in composition 1A, which is a formula containing 5% of the conventional extract in an oil-in-water/Arlacel/Myrj emulsion containing 5% parleam and 15% volatile silicone. The effect of this composition 1A is compared with that of a placebo: composition 2A which corresponds to the excipient: oil-in-water/Arlacel/Myrj emulsion containing 5% parleam and 15% volatile silicone.

The change in the signs and symptoms of atopic dermatitis of the patients treated, in comparison with the placebo effect, is observed contra-laterally (p=0.008, Wilcoxon test).

Composition 1A containing the “conventional extract” of Vitreoscilla filiformis at 5% did not have a significant action on the dryness of the skin of the individuals tested.

A second new clinical study (study 2 carried out under the same conditions as the previous study and by the same team of experimenters) intended to evaluate the intraindividual effectiveness of a cream containing 5% of bacterial extract prepared according to example 1, showed a specific effectiveness on the states of dryness found in slight to moderate atopy.

The extracts formulated in composition 1B, which is a formula containing 5% of bacterial extract according to the invention (obtained according to example 1) in an oil-in-demineralized water Arlacel/Myrj emulsion containing 5% parleam, 15% cyclopentasiloxane, 3% glycerol and 2% petroleum jelly. The effect of this composition 1B is compared with that of a placebo: composition 2B which corresponds to an oil-in-water from La Roche Posay Arlacel/Myrj emulsion containing 5% parleam, 15% cyclopentasiloxane, 3% glycerol and 2% petroleum jelly.

Composition 1B comprising the bacterial extract according to the invention is applied twice a day and is very well tolerated; the difference compared with the above study lies in the number of visits and the analysis of the persistence of the activity of the product.

The composition significantly decreased the intensity of excoriation and lichenification and the repercussion thereof on the pruritis of atopic dermatitis in the patients, compared with the effect of the contra-lateral plabeco (p=0.0041, Fisher test). The therapeutic effectiveness was observed for the 15 days which followed application.

This superiority in terms of effectiveness compared with the previous study is due to its specificity in acting on the dryness of the skin observed in these patients. These extracts cultured on water from La Roche Posay significantly decreased the signs and symptoms due to the dryness of the skin of the patients compared with the contra-lateral placebo effect (p=0.01, Fisher test).

### EXAMPLE 3

### Clinical Trials on Individuals Suffering from Seborrheic Dermatitis

The third clinical study evaluated the effectiveness of an aqueous-alcoholic lotion containing 5% of extract of Vitreoscilla filiformis, prepared according to example 1, on seborrheic dermatitis. The composition of the formula is a mixture of polyethylene glycol stearate, aminomethyl propanol and cyclopentadimethylsiloxane prepared in distilled water.

In this study, the products tested, applied once a day, were very well tolerated.

These products significantly decreased the erythemato squamous lesions and the pruritis of individuals with seborrheic dermatitis of the scalp, in the patients compared with the placebo effect (p<0.0001, Chi-Squared). The effectiveness was observed for the 15 days following application.

It is interesting to note that a decrease in desquamation lesions (scores) of 58.6% is specifically apparent for the aqueous-alcoholic lotion containing the bacterial extract according to the invention only, the placebo aqueous-alcoholic lotion causing only a slight variation in the desquamation scores from D1 and J28 (decrease of 13.5%).

These statistical analyses underline the fact that the treatment with the bacterial extract according to the invention enables a significant decrease in the desquamation score during the treatment phase (this being compared with the placebo group (p<0.0001)).

If the persistence of the activity of the lotions is analyzed, it is noted that the desquamation, even after the treatment has been stopped, varies only slightly at D35 compared with D28 (no significant difference from D28 and D35 for the bacterial extract according to the invention (p=0.1698)), showing a long-lasting beneficial effect.

### EXAMPLE 4

### Topical Compositions

Facial care milk for dry skin:

| | |
|---|---|
| Magnesium chloride | 3.00 |
| Calcium ascorbate | 3.00 |
| Bacterial extract according to | 5.00 |
| Example 1 | |
| Glyceryl stearate | 1.00 |
| Cetylsteryl alcohol/oxyethylenated | 3.00 |
| cetyl stearyl alcohol comprising 30 moles | |
| EO (Sinnowax AO ® marketed by HENKEL) | |
| Cetyl alcohol | 1.00 |
| Dimethicone (DC 200 Fluid ® | 1.00 |
| marketed by DOW CORNING) | |
| Liquid petroleum jelly | 6.00 |
| Isopropyl myristate (Estol IMP 1514 ® | 3.00 |
| marketed by UNICHEMA) | |
| Antioxidant | 0.05 |
| Glycerol | 20.00 |
| Conservative | 0.30 |
| Water | qs 100 |

Facial care milk for dry skin:

| | |
|---|---|
| Magnesium ascorbate | 3.00 |
| Blackcurrent seed oil | 4.00 |
| Borage oil | 4.00 |
| Bacterial extract according to | 5.00 |
| Example 1 | |
| Glyceryl stearate | 1.00 |
| Cetylsteryl alcohol/oxyethylenated | 3.00 |
| cetyl stearyl alcohol comprising 3 moles | |
| EO (Sinnovax AO ® marketed by HENKEL) | |
| Cetyl alcohol | 1.00 |
| Dimethicone (DC 200 Fluid ® | 1.00 |
| marketed by Dow Corning) | |
| Liquid petroleum jelly | 6.00 |
| Isopropyl myristate (Estol IMP 1514 ® | 3.00 |
| marketed by Unichema) | |
| Glycerol | 20.00 |
| Conservative | 0.30 |
| Water | qs 100 |

Emollient body lotion:

| | |
|---|---|
| Mineral oil | 8.0% |
| Isopropyl palmitate | 5.0% |
| Polyglyceryl-3 diisostearate | 4.0% |
| Octyldodecanol | 4.0% |
| Carbomer | 0.3% |
| Bacterial extract according to | 2.0% |
| Example 1 | |
| Sodium cocoylglutamate | 2.0% |
| 10% sodium hydroxide | 1.2% |
| Preservative | 0.5% |
| Fragrance | 0.5% |
| Water | qs 100% |

Anti-dandruff shampoo:

| | |
|---|---|
| Sodium lauryl sulfate | 7.0% |
| Cocamidopropylbetain | 2.0% |
| Sodium lauryl sulfosuccinate | 2.0% |
| Bacterial extract according to | 4.0% |
| Example 1 | |
| Sodium chloride | 1.0% |
| Preservatives | 0.5% |
| Fragrance | 0.5% |
| Water | qs 100% |

Cream for dry skin:

| | |
|---|---|
| Arachidyl behenyl alcohol/ | 3.0% |
| arachidylglusoside | |
| Isohexadecane | 7.0% |
| Sweet almond oil | 3.0% |
| Shea butter | 2.0% |
| Glycerol | 5.0% |
| Bacterial extract according to | 3.0% |
| Example 1 | |
| BHT | 0.05% |
| Methyl POB | 0.1% |
| Propyl POB | 0.05% |
| Water | qs 100% |

Cream for very dry skin:

| | |
|---|---|
| Bacterial extract according to | 1.5% |
| Example 1 | |
| Glyceryl stearate and | 5.0% |
| PEG 100 stearate | |
| Isohexadecane | 8.0% |
| Shea butter | 5.0% |
| Glycerol | 3.0% |
| Carbopol 981 0.2% | 0.2% |
| Lubragel | 5.0% |
| Phenoxyethanol | 1.0% |
| Sodium hydroxide | qs pH6 |
| BHT | 0.05% |
| Dc 1503 | 1.0% |
| Water | qs 100% |

Each patent, patent application, publication, text and literature article/report cited or indicated herein is hereby expressly incorporated by reference in its entirety.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A cosmetic/dermatological regime or regimen for treating the signs associated with dryness of keratin minerals, comprising administering to an individual in need of such treatment, a thus effective amount of at least one extract of a non-photosynthetic and non-fruiting filamentous bacterium cultured in a medium which comprises at least one non-sulfurous mineral and/or thermal water.

2. The cosmetic/dermatological regime or regimen as defined by claim 1, said non-photosynthetic and non-fruiting filamentous bacterium comprising Vitreoscilla filiformis.

3. The cosmetic/dermatological regime or regimen as defined by claim 1, said at least one non-sulfurous mineral and/or thermal water comprising that from La Roche Posay.

4. The cosmetic/dermatological regime or regimen as defined by claim 1, said non-photosynthetic and non-fruiting filamentous bacterium comprising those of the genera Beggiatoa, Vitreoscilla, Flexithrix or Leucothrix.

5. The cosmetic/dermatological regime or regimen as defined by claim 1, said non-photosynthetic and non-fruiting filamentous bacterium comprising:
Vitreoscilla filiformis (ATCC 15551);
Vitreoscilla beggiatoides (ATCC 43181);
Beggiatoa alba (ATCC 33555);
Flexithrix dorotheae (ATCC 23163);
Leucothrix mucor (ATCC 25107); or
Sphaerotilus natans (ATCC 13338).

6. The cosmetic/dermatological regime or regimen as defined by claim 1, said at least one non-sulfurous mineral and/or thermal water having a total mineral content ranging from 400 to 900 mg/l.

7. The cosmetic/dermatological regime or regimen as defined by claim 1, said at least one non-sulfurous mineral and/or thermal water having a total concentration of carbonates and of bicarbonates of at least 150 mg/l and a concentration of silicon oxide of at least 6 mg/l.

8. The cosmetic/dermatological regime or regimen as defined by claim 1, said at least one non-sulfurous mineral and/or thermal water comprising that from Vittel, from the Vichy basin, from Uriage, from La Bourboule, from Enghien-les-Bains, from Saint-Gervais-les-Bains, from Néris-les-Bains, from Allevard-les-Bains, from Digne, from Maizières, water from Neyrac-les-Bains, from Lons-Ie-Saunier, from Eaux-Bonnes, from Rochefort, from Saint Christau, from Les Fumades or from Tercis-les-Bains.

9. The cosmetic/dermatological regime or regimen as defined by claim 6, said at least one non-sulfurous mineral and/or thermal water having a concentration of calcium ions of at least 100 mg/l.

10. The cosmetic/dermatological regime or regimen as defined by claim 1, said at least one extract comprising from 0.001 to 20% by weight of the total weight of a composition comprised thereof.

11. The cosmetic/dermatological regime or regimen as defined by claim 1, said non-photosynthetic and non-fruiting filamentous bacterium having been cultured in a medium comprising autolyzed yeast extract, plant peptone, anhydrous glucose, Heller microelements and calcium chloride.

12. The cosmetic/dermatological regime or regimen as defined by claim 1, said at least one extract comprising Se, Sr and Zn values.

13. The cosmetic/dermatological regime or regimen as defined by claim 1, comprising topically applying said at least one extract onto the affected keratin materials area of such individual.

14. The cosmetic/dermatological regime or regimen as defined by claim 1, including treating a state of dryness of the skin, a squamous state and/or itching and/or tautness associated with dry skin.

15. A cosmetic/dermatological regime or regimen for treating a skin disorder related to a deficiency in excretion and/or secretion of sebum and/or physiologically restoring a suitable state of hydration of the stratum corneum and/or treating hyposeborrheic dry skin and/or stimulating sebum production, comprising administering to an individual in need of such treatment, a thus effective amount of at least one extract of a non-photosynthetic and non-fruiting filamentous bacterium cultured in a medium which comprises at least one non-sulfurous mineral and/or thermal water.

16. A cosmetic/dermatological regime or regimen for preventing and/or reducing wrinkles associated with dryness of the skin, comprising administering to an individual in need of such treatment, a thus effective amount of at least one extract of a non-photosynthetic and non-fruiting filamentous bacterium cultured in a medium which comprises at least one non-sulfurous mineral and/or thermal water.

17. A cosmetic/dermatological regime or regimen for treating dry keratin fibers and/or improving the comfort of dry skin and/or dry scalp, comprising administering to an individual in need of such treatment, a thus effective amount of at least one extract of a non-photosynthetic and non-fruiting filamentous bacterium cultured in a medium which comprises at least one non-sulfurous mineral and/or thermal water.

18. The cosmetic/dermatological regime or regimen as defined by claim 1, comprising combating the dull and/or lifeless appearance of the skin and/or keratin fibers.

19. The cosmetic/dermatological regime or regimen as defined by claim 1, comprising treating constitutional or acquired, non-pathological dry skin.

20. The cosmetic/dermatological regime or regimen as defined by claim 1, comprising treating constitutional non-pathological dry skin selected from senile skin and common xerosis.

21. A cosmetic/dermatological regime or regimen for treating atopic dermatitis, atopic xerosis, seborrheic dermatitis and hyperkeratosis, comprising administering to an individual in need of such treatment, a thus effective amount of at least one extract of a non-photosynthetic and non-fruiting filamentous bacterium cultured in a medium which comprises at least one non-sulfurous mineral and/or thermal water.

22. A cosmetic/dermatological regime or regimen for preventing and/or treating dry keratin materials, dry and/or delicate skin, and/or for treating hyposeborrheic dry skin, and/or for preventing and/or treating itching and/or tautness and/or for stimulating sebum production, and/or for the treatment and/or prevention of skin disorders and/or disorders of the pilosebaceous unit related to a deficiency in excretion and/or secretion of sebum, and/or for preventing and/or treating dry or brittle keratin fibers, and/or for preventing and/or reducing wrinkles associated with dryness of the skin, comprising administering to an individual in need of such treatment, a thus effective amount of at least one extract of a non-photosynthetic and non-fruiting filamentous bacterium cultured in a medium which comprises at least one non-sulfurous mineral and/or thermal water.

23. A cosmetic/dermatological composition useful for treating the signs associated with dryness of keratin materials, comprising at least one extract of a non-photosynthetic and non-fruiting filamentous bacterium cultured in a medium which comprises at least one mineral and/or thermal water and at least one other active agent selected from the group consisting of vitamins B3, B5, B6, B8 C, E or PP, carotenoids, curcuminoids and niacin.

24. A cosmetic/dermatological composition useful for treating the signs associated with dryness of keratin materials, comprising at least one extract of a non-photosynthetic and non-fruiting filamentous bacterium cultured in a medium which comprises at least one mineral and/or thermal water and at least one other active agent selected from the group consisting of desquamating agents; moisturizing agents; anti-glycation agents; anti-inflammatories or calmatives; agents for stimulating keratinocyte proliferation and/or differentiation; anti-dandruff agents; and anti-microbial agents.
